(19) United States
(12) Patent Application Publication
(10) Pub. No.: US 2026/0041588 A1
(43) Pub. Date: Feb. 12, 2026

(54) Elevation Apparatus, System and Methods of Use

(71) Applicant: Sun Scientific, Inc., Dobbs Ferry, NY (US)

(72) Inventors: Sundaram Ravikumar, Braircliff Manor, NY (US); Vikram Ravikumar, New York, NY (US); Guy Osborne, Trumball, CT (US)

(73) Assignee: Sun Scientific, Inc., Dobbs Ferry, NY (US)

(21) Appl. No.: 18/875,453

(22) PCT Filed: Jun. 17, 2023
(86) PCT No.: PCT/US2023/025642

§ 371 Date: Dec. 16, 2024

Related U.S. Application Data
(60) Provisional application No. 63/353,438, filed on Jun. 17, 2022.

(51) Int. Cl. A61F 13/06; A61F 5/34; A61F 13/0203

(52) U.S. Cl. CPC A61F 13/069; A61F 5/34; A61F 13/0203

(57) ABSTRACT

An elevation apparatus to elevate a bony prominence of a user such as a heel, ankle, foot, metatarsal, elbow, hip, sacrum, shoulder blade or other bones. The elevation apparatus including a permeable skin-compatible adhesive layer, an absorbent layer, a protective layer and at least one inflatable or prefilled air bladder including at least one aperture configured for the selected bony prominence.

### RELATED APPLICATIONS

The present application claim priority from PCT application Serial No. PCT/US23/025642 entitled ‘Elevation Apparatus, System and Methods of Use” filed Jun. 17, 2023 claiming priority from U.S. provisional application Ser. No. 63/353,438 filed Jun. 17, 2022, the entire contents of which is hereby incorporated herein by reference for all purposes.

### BACKGROUND OF THE INVENTION

### I. Field of the Invention

The subject invention is directed generally to an apparatus and system for elevating a bony prominence on the body for a user to reduce or prevent pressure sores and skin ulcers.

### II. Background of the Related Art

A pressure ulcer (injury) is a localized injury to the skin and soft tissue over a bony prominence caused by external forces and is one of the most resistant chronic wounds. It can be caused by friction with clothing or caused by various ailments of the patient. Some pressure ulcers are also commonly referred to as “bed sores”. These pressure skin ulcers can form on patients in nursing homes and hospitals or even at home if the person is at rest for a longer period of time. Areas where bones are close to the surface (called “bony prominences”) and areas that are under the most pressure are at greatest risk for developing pressure ulcers or sores. Certain bony prominences are more common to pressure ulcers or bed sores such as the ball of the foot, below the metatarsal bones, ankle, hip, sacrum or tail bone, knee including front and back and sides, elbow, and back including shoulder blades.

It is known to treat pressure ulcers and sores after they develop by various dressings but preventing them or reducing the occurrence is not known. Very often the patients who have these pressure ulcers or bed sores also have other underlying conditions such as diabetes and circulation issues which contribute to the pressure ulcers or bed sores not clearing up quickly. Typically, the patients in a nursing home, hospital or on bed rest at home will not recover once the pressure ulcer or bed sore has developed. Again, these patients typically have pressure ulcers on their ankles, sacrum and hips as well as other bony prominences.

When a patient is not ambulatory, the patient faces an elevated risk factor of pressure ulcers or bed sores due to immobility and pressure including increased pressure on bony prominences such as the ankle, heel, metatarsal bone, elbow, back and hip, sacral bone, among other body parts. Such pressure ulcers typically develop on either side of the lower leg, around or on the ankle bone that protrudes as well as the hip bone and tail bone. Such pressure ulcers and bed sores are difficult to heal and even if they do partially heal will often recur over and over, eventually developing infections. Further, skin ulcers or bed sores may develop around the knee, along the sides and on the front of the knee as well as the back of the knee for a person sitting in a wheelchair most of the day.

In some studies it has been found that seventy-five percent of the pressure ulcers or bed sore injuries occur around the pelvic girdle, most often at the ischium, greater trochanter, and sacrum. Apart from ischemia, other factors that prevent normal healing from occurring include poor nutrition, infection, edema, persistent moisture, fecal and urinary soiling, and shearing forces. Specific risk factors for sacral pressure ulcers and bed sores include lying in the supine position and fecal incontinence. Again, merely placing a pillow under the tail bone does not alleviate the risk as the individual will dislodge the pillow due to any movement in the bed or chair. Treating the bleeding or oozing current pressure ulcer or bed sore with a bandage, dressing does not deal with the pressure point itself.

Pressure ulcers or bed sores can be caused by a combination of pressure, friction and shear. Constant pressure on any part of the body can lessen the blood flow to skin and the underlying tissue. Without these essential oxygen and other nutrients to skin and tissue the skin and nearby tissue are damaged and might eventually die. For people with limited mobility, this kind of pressure tends to happen in areas that aren't well padded with muscle or fat and that lie over a bone, such as the heel, ankle, ball of the foot or metatarsal, spine, tailbone or sacrum, shoulder blades, hips, and elbows which are all bony prominences. Also contributing to pressure ulcers and bed sores is friction which occurs when the skin rubs against clothing or bedding. Such friction can make fragile skin more vulnerable to injury, especially if the skin is also moist. Finally, shear contributes such as when two surfaces move in the opposite direction. For example, when a person is in bed they can slide down such as when the sacral bone or tailbone moves down, the skin over the bone might stay in place-essentially pulling in the opposite direction. All of these contribute to causing pressure ulcers and bed sores.

Pressure ulcers and bed sores, beyond common bleeding and pain, can result in more extreme injuries such as cellulitis, infection, cancer and even sepsis or death. Cellulitis is an infection of the skin and connected soft tissues which can cause warmth, inflammation and swelling of the affected area. People with nerve damage often do not feel pain in the area affected by cellulitis. An infection from a pressure ulcer or bed sore can burrow into joints and bones. Joint infections (septic arthritis) can damage cartilage and tissue. Bone infections (osteomyelitis) can also reduce the function of joints and limbs. In some instances, the pressure ulcer and bed sore can result in cancer. Long-term, nonhealing wounds (Marjolin's ulcers) can develop into a type of squamous cell carcinoma. Also in some instances pressure ulcers or bed sores can lead to sepsis and even death. Therefore a need exists to treat, reduce and even prevent pressure ulcers and bed sores.

Many of the individuals who develop pressure ulcers and bed sores also have lymphedema, also known as lymphatic obstruction, which is a condition of localized fluid retention and tissue swelling, and is caused by a compromised lymphatic system. Treatment for lymphedema varies depending on the severity of the edema and the degree of fibrosis of the affected limb. The most common treatments for lymphedema are manual compression lymphatic massage, compression garments or bandaging. Elastic compression garments are typically worn by persons with lymphedema on the affected limb following complete decongestive therapy to maintain edema reduction. These individuals who develop pressure ulcers and bed sores who also have lymphedema do not recover from the pressure ulcers and bed sores and instead they linger on the skin for months or longer.

Diabetic ulcers may affect the foot and ankle area resulting in exudating wounds. These ulcers may be on the bottom or under sole of the foot, ball of the foot, along the metatarsal bones, on the toes, between the toes, on the top of the foot, on the arch of the foot and on the ankles and heels of the foot. Diabetics may have more foot ulcers sometimes related to a complication called neuropathy that causes the individual to lose feeling in their feet. A scrape, cut or puncture in the skin can turn into an ulcer, but the individual might not know it's there if they have neuropathy and do not feel the wound or pain from the wound on the ankle or heel or foot area. Ulcers can lead to infections and in extreme cases the infection will not heal resulting in partial amputation of the individual's foot or toe(s). It is estimated that about 15% of people with diabetes in the United States will get a foot or toe ulcer and around 14% to 24% of people with diabetes in the U.S. need an amputation after they get an ulcer. Diabetic foot ulcers are the leading cause of foot amputations. As of 2020, of the estimated 537 million people worldwide who had diabetes about 19% to 34% will develop a diabetic foot ulcer in their lifetime. Unfortunately, of those individuals who develop a diabetic foot ulcer, approximately 20% of them will require lower-extremity amputation, either minor (below the ankle), major (above the ankle), or both. More than 80 percent of amputations start with a foot ulcer. Even more extreme, of those who develop a diabetic foot ulcer about 10% will die within one year of their first diabetic foot ulcer diagnosis. Further, foot ulcers can happen to many people but might be more common in Black, Native American and Hispanic people. If the individual has an eye, kidney or heart disease related to diabetes, they are also at a higher risk. Of those about 15% of people with diabetes with an ulcer, the ulcer is typically on the bottom of their foot. Some of those people will be hospitalized because of complications.

Individuals are also at a higher risk of getting foot and toe ulcers if they have any of the following conditions: blood circulation issues, heart disease, obesity, a foot condition like a bunion or hammertoe, kidney disease, and some lifestyle behaviors of using tobacco and alcohol. Ulcers are open wounds in the skin that can take on almost any shape. Some shapes are more common than others on specific parts of the body. For example, ulcers on the foot or toe might be shaped like a crater or a wedge. The ulcer can go down even into the bone resulting in amputation or even death.

Neurotrophic ulcers occur primarily in people with diabetes, although they can affect anyone who has impaired sensation in their feet. They can be found anywhere on the feet but typically develop on the parts of the feet and toes that are most sensitive to weight (pressure points) such as the ankle, heel and ball on the bottom of the foot. While neurotrophic or diabetic ulcers don't cause pain based on the lack of feeling on the foot due to neuropathy, this can be worse because the individual will not realize they have an ulcer until it is weeping or exudating. Further, autonomic neuropathy is associated with decreased production of sweat and components of the natural moisturizing factor leaving the skin dry that leads to disruption of the epidermal barrier resulting in dryness and the presence of fissures or cracks that may become a portal of entry for bacteria on the foot and even in between the toes. Areas with blisters, cuts, scratches, redness, or hemorrhage are indicative of local damage from local trauma, friction, or shear commonly caused by walking barefoot and poorly fitting footwear. Callus formation is a reaction to increased local pressure and heralds impending skin breakdown and ulceration. Yet another complication is a fungal infection which is not uncommon in the feet of diabetics. Diabetic foot ulcers that aren't treated promptly can cause major complications. In addition to being significantly more susceptible to ulceration, a diabetic patient will also generally take a significantly longer period for such ulceration to heal. It is not uncommon for it to take 10-12 weeks for an ulcer on the foot, heel or ankle of a diabetic patient to heal even when using a cast or other more permanent treatment or therapy where the individual may be on crutches with complete off-loading of any weight or pressure on the foot. In comparison, such an ulcer would likely heal in less than seven days in a healthy individual. A need exists for an elevation apparatus that is easy to apply by the individual alone or by a medical professional to prevent pressure sores and if a sore or ulcer is already present exudates the ulcer or wound, improves healing times, reduces complications and assist in healing overall.

Arterial ulcers may be caused by a condition called peripheral arterial disease which reduces blood flow to your extremities. When this happens the foot tissue may start to die. The ulcers that form from reduced blood flow are called arterial ulcers and typically form near the ankle of the foot. Arteries are blood vessels that transport blood from the heart to the rest of the body, including the feet and toes. Anyone can get an arterial ulcer, but individuals who smoke or have diabetes, high blood pressure or high cholesterol are at higher risk. Unlike neurotrophic ulcers, arterial ulcers can form on many parts of the foot most notably on the bony parts of the feet such as the ball of the foot, along the metatarsal bones, heel and ankles area that rub against bed sheets, socks or shoes, and even furniture such as the couch or chair.

Both diabetic ulcers and arterial ulcers are wounds or open sores that do not heal, or otherwise recur repeatedly, and cause persistent swelling as well as burning, itching, irritation and discoloration of the skin. Conventional treatment and therapy for such ulcers generally includes topical protection of the wound, as well as compression and antimicrobial treatment of the affected area. Other treatment plans for the foot, heel, ankle, hip and sacral area include draining the ulcer or wound site, orthotics to try to reduce or shift pressure points on the foot, removing pressure from the area by wearing a cast, elevation of the target bony prominence for extended periods of time (which is difficult for individuals working), debridement and removal of infected tissue, shaving or removing bones that in infected, tenotomy which is removal of scar tissue that has developed at the ulcer or wound site, and even reconstructive surgery using skin grafts to replace the infected ulcer or would site.

Many of the current treatment options including the use of current known devices, apparatus, bandages, stocking, hosiery and the like just treat the bleeding or oozing pus and do nothing to prevent or reduce the occurrence of the pressure ulcers or bed sores. Other treatments including placing a pillow under the individual's heel or ankle but once the patient moves even a tiny bit the body part can have contact with the bed again and even if the pillow is placed in the correct position by a trained caretaker, the pillow itself may form or contribute to a pressure ulcer or bed sore. Currently known elevation apparatus and devices are not successful in preventing pressure ulcers on the bony prominences of the foot, heel, ankle, hip and sacrum.

The venous pressure at the ankle of a subject who is lying supine is around 10 mmHg, but on standing this will rise considerably due to an increase in hydrostatic pressure (equivalent to the weight of a vertical column of blood stretching from the point of measurement to the right auricle of the heart). During walking, as the foot is dorsally flexed, the contraction of the calf muscle compresses the deep veins and soleal sinuses thereby emptying them of blood. As the foot is plantarly flexed, the pressure in the veins falls, the proximal valves close, and the veins are refilled by blood passing through the perforators from the superficial system. During this cycle, in a normal leg, the distal valves of the deep veins and the valves of the perforators will ensure that the expelled blood can go in only one direction—upwards, back to the head. Thus, any bandage or dressing placed on the pressure ulcer or bed sore has to take into account these venous pressures as well so that the bandage or dressing is not too tight or loose.

Again, many of the individuals suffering from pressure ulcers or bed sores also have CVI which occurs when these valves become damaged and allow the blood to leak back downward in the opposite direction. Such valve damage may occur as the result of aging, extended sitting or standing, or a combination of aging and reduced mobility. When the veins and valves become weakened and the blood does not properly flow up to the heart, blood pressure in the veins of the lower extremities can stay elevated for long periods of time, leading to CVI. This condition is more common in older individuals, and if not properly treated, can lead to burst capillaries, local tissue inflammation, internal tissue damage, varicose veins, ulcers, and open sores on the skin's surface. The burst capillaries can be seen on the patient's leg including the ankle, knee, thigh, hip, sacrum, back and groin. CVI contributes to longer recovery time from a pressure ulcer or bed sore.

Beyond pain and discomfort, along with potential of up to cancer and sepsis, the prevalence of pressure ulcers and bed sores also have economic burdens. Again, after a few hours, pressure on the bony prominences specifically the heel, ankle, sacrum and the hip cause bed sores and ulcerations secondary to constant pressure and blood supply to the skin on this area. Initially just redness and as immobility continues over time, redness becomes ulcer and tends to go to the bone. Once an ulcer forms it takes months to heal and cause discomfort and pain, as well as huge economic burden on the nursing homes and hospitals. Economic strain from the cost of materials to change the dressings is compounded by economic cost for the time for medical staff to change the multitude of dressings on these pressure ulcers and bed sores. One patient in a nursing home or hospital may have for instance 4-5 pressure ulcers and bed sores which all need change of bandage and dressing, which is then multiplied by the number of residents in a nursing home or assisted living facility as well as hospitals. This can cost a full time medical person just moving from patient and resident and so on all day throughout every shift. Thus, a need exists to treat, reduce or even prevent pressure ulcers and bed sores.

Currently there are a number of tapes and bandages that are available for application to a wound site or ulcer but the anatomy and structure of the foot, heel, ankle, hip and sacrum area causes problems with adhesion, pressure complicating any treatment and healing, as well as leakage or exudating of the ulcer onto the individual's sock, clothing, bedsheets, couch and the like. A need exists for a wound dressing on the foot, heel, ankle, hip and sacrum for ulcers or any type of wound. Notably, common wounds, scrapes, cuts and the like on these bony areas take longer to heal even in healthy individuals again due to the anatomy and structure of the bony prominences.

A need exists for a wound dressing for any wound on the foot regardless of whether the individual has diabetes, circulation issues or is a healthy individual with a foot wound. As of 2015, costs related to diabetic foot ulcers alone were greater than $1 billion annually and rising yearly. As of 2022 it was estimated that overall diabetes care in the U.S. accounts for an estimated $273 billion in direct and $90 billion in indirect costs annually. Foot complications such as ulcers and infections represent a major source of costs among people with diabetes, leading to higher rates of hospital admission, emergency department visits, outpatient visits and home health care utilization, and excess annual expenditures of 50% to 200% above the baseline cost of diabetes-related care. Advanced-stage ulcers wherever they are located including the heel, ankle, hip and/or sacrum cost upwards of $50,000 per wound episode, and direct costs of major amputation are even higher. These numbers likely underestimate the true economic burden of diabetic foot ulcers and all pressure ulcers given out-of-pocket expenses, loss of productivity, and decreased employment associated with such ulcers or open sores. A need exists for an apparatus to elevate such bony prominences as well as a treatment plan to improve healing times and outcomes while reducing complications such as infections and amputations for such ulcers as well as arterial ulcers, while also improving function and quality of life and reducing health care costs.

Thus a need exists for an apparatus to elevate, treat, reduce and even prevent pressure ulcers and bed sores which elevates the bony prominences while also treating any current pressure ulcers or bed sores at the same time. A further needs exists for an apparatus which will not shift or move when the individual moves while in bed, a chair or otherwise partially or completely immobile. Yet another need exists for an apparatus that can be used when the individual walks again without shifting or moving on the skin. Yet another need exists for an apparatus which includes a pre-filled or partially filled air bladder to elevate the bony prominence as well as the ability to distribute the pressure evenly across the underlying anatomy resulting in optimal compression and comfort.

The apparatuses, system and methods of the subject invention provide benefits and advantages that may overcome a number of problems with respect to known technologies, particularly the problems that arise due to the difficulty of applying current dressings over a pressure ulcer or bed sore and elevating with pillows or other known devices. Those skilled in the art will readily appreciate that it would be beneficial to provide an elevating apparatus for preventing, treating, reducing, assisting in healing and recovery or even preventing pressure ulcers and ulcers on bony prominences. Other uses for the inventive apparatus may be envisioned.

### SUMMARY OF THE INVENTION

The subject invention is directed to an apparatus, system and method of use to treat, reduce or even prevent pressure ulcers and/or bed sores on an individual near or on the bony prominences of a foot, ankle, heel, hip bone, elbow, should blades and/or sacrum.

According to one aspect of the invention, the apparatus includes a flexible member adapted to adhere to the skin of an individual, including a pre-filled air bladder or an air bladder configured to be partially filled once adhered to the skin. The flexible member may be a foam, sponge or dressing on or around a bony prominence to elevate the target body part when the individual is immobile for a period of time or to be a layer or barrier between the skin and clothing, linens, furniture or other forces. These and other aspects of the contacts of the subject invention will become more readily apparent from the following description taken in conjunction with the drawings.

### BRIEF DESCRIPTION OF THE DRAWINGS

So that those having ordinary skill in the art to which the subject invention pertains will more readily understand how to make and use the apparatuses of the subject invention, preferred embodiments thereof will be described in detail herein below with reference to the drawings, wherein:

FIGS. 1A-1D are a perspective view (1A), side top view (1B), bottom view (1C), and top view (1D) of one embodiment of the present invention including an apparatus to elevate a sacrum bone;

FIG. 2 is an exploded view of FIGS. 1A-1D;

FIGS. 3A-3D are a perspective view (3A), side top view (3B), bottom view (3C) and top view (3D) of another embodiment of the present invention including an apparatus to elevate a sacrum bone having an inflatable bladder with an inlet valve and where the two bladders are joined;

FIGS. 4A-D are a perspective view (4A), side top view (4B), bottom view (4C), and top view (4D) of another embodiment of the present invention including an apparatus to elevate a hip bone or elbow and one side of an ankle or a heel;

FIG. 5 is an exploded view of the embodiment of FIGS. 4A-4D;

FIGS. 6A-6D are a perspective view (6A), side top view (6B), bottom view (6C), and top view (6D) of another embodiment of the present invention including an apparatus to elevate a metatarsal bone or ball of the foot or any slightly elongated bony prominence;

FIG. 7 is an exploded view of the embodiment of FIGS. 6A-6D;

FIGS. 8A-8D are a perspective view (8A), side top view (8B), bottom view (8C), and top view (8D) of yet another embodiment of the present invention including an apparatus to elevate a heel of a user;

FIG. 9 is an exploded view of the embodiment of FIGS. 8A-8D;

FIGS. 10A-10D are a perspective view (10A), side top view (10B), bottom view (10C), and top view (10D) of yet another embodiment of the present invention including an apparatus to elevate a heel and each side of an ankle of a user including three separate pre-filled air bladders;

FIG. 11 is an exploded view of the embodiment of FIGS. 10A-10D;

FIGS. 12A-12D are a perspective view (12A), side top view (12B), bottom view (12C), and top view (12D) of yet another embodiment of the present invention including an apparatus to elevate a heel and each side of an ankle of a user including one pre-filled air bladder;

FIG. 13 is an exploded view of the embodiment of FIGS. 12A-12D;

FIG. 14 is a perspective view of yet another embodiment of the present invention including an apparatus to elevate a heel and each side of an ankle of a user including one inflatable bladder with an inlet valve; and

FIGS. 15A-15D are a flow diagram of the method of applying an apparatus of FIGS. 8-9 on the heel and secured to the bottom of the foot and sides of the ankle.

### DETAILED DESCRIPTION OF THE PREFERRED EMBODIMENTS

Preferred embodiments of the subject invention are described below with reference to the accompanying drawings, in which like reference numerals represent the same or similar elements. One of ordinary skill in the art would appreciate that while the apparatuses discussed herein relate to elevation and therapy of certain bony prominences such as the ankle, heel, hip, sacrum, and metatarsal bone, however the scope of the invention is not limited to those exemplary body parts and applications and may be sized and shaped for the anatomical portion for which elevation and therapy is needed.

The subject invention provides compression to a patient's limbs, including the bony prominences, including for example, the ankle, heel, metatarsal bone, hip, tailbone or sacrum, as well as knee and shoulder blades, in a manner that is simpler and more convenient than current apparatus and systems. Any limb or body part may be compressed by the instant apparatus such as for instance a foot, calf, thigh, knee, leg, hip, buttocks, waist, torso, ribs, shoulder, arm, hand, fingers, neck, head or the like.

The subject invention provides an apparatus for elevating a bony prominence to treat, reduce and even prevent pressure ulcers and bed sores. The inventive apparatus creates elevation and lessens pressure of the skin at a bony prominence in a manner that allows for consistent and constant relief when the user is immobile in bed or a chair as well as when moving such that the apparatus does not move once adhered or connected to the skin of the user. The inventive apparatus provides a safe, comfortable, convenient, effective, self-application by the patient or user.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an” and “the” include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

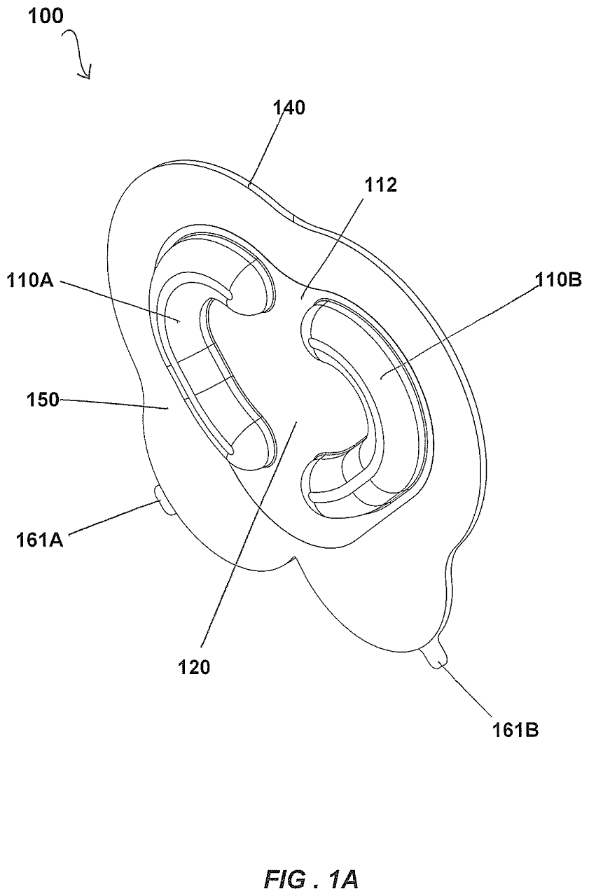

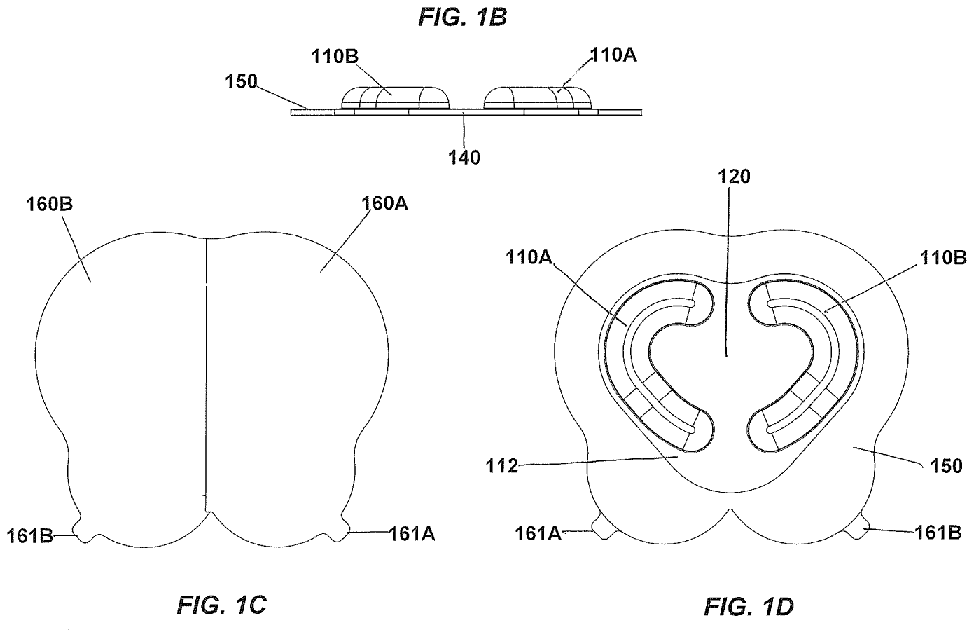

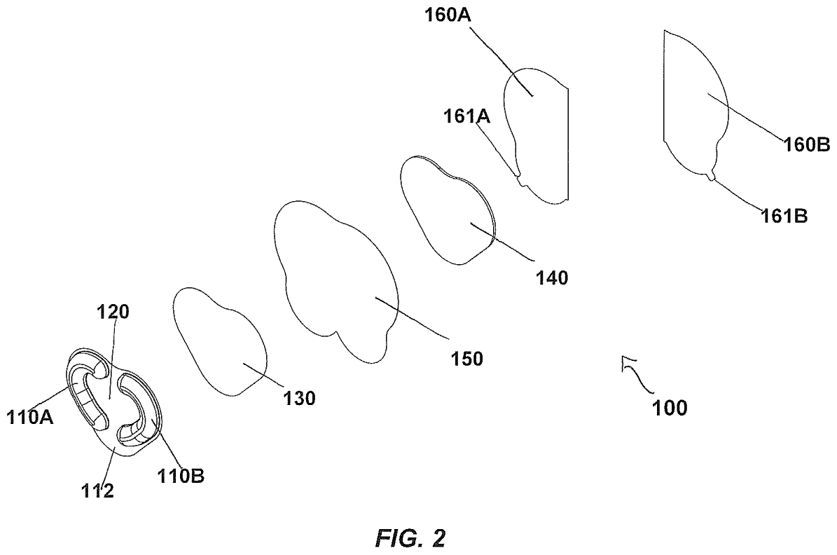

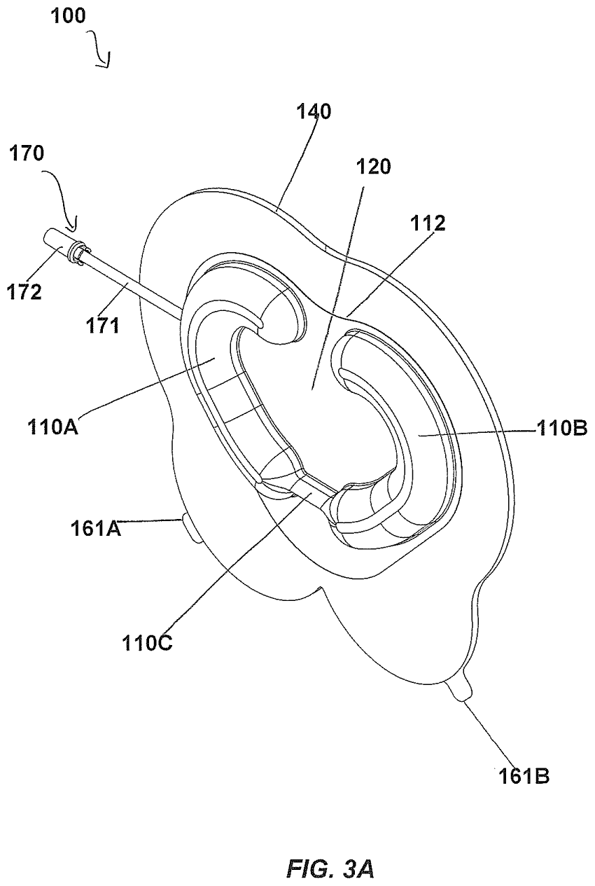

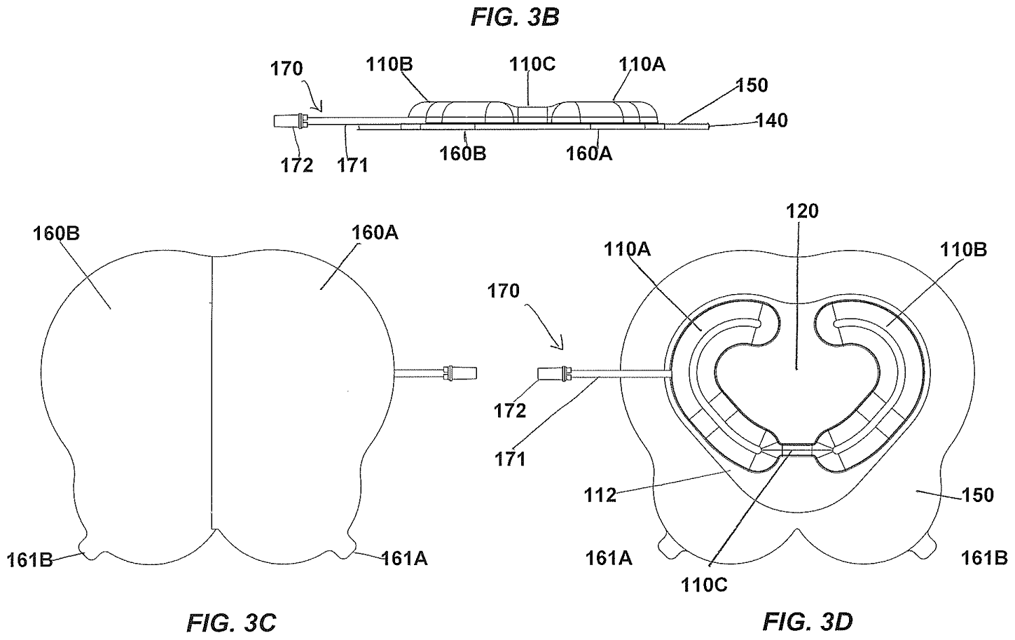

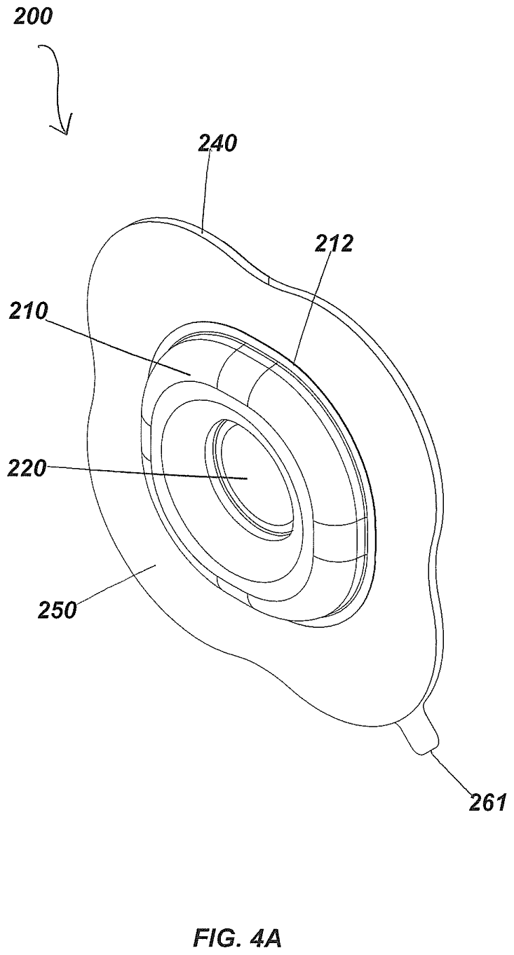

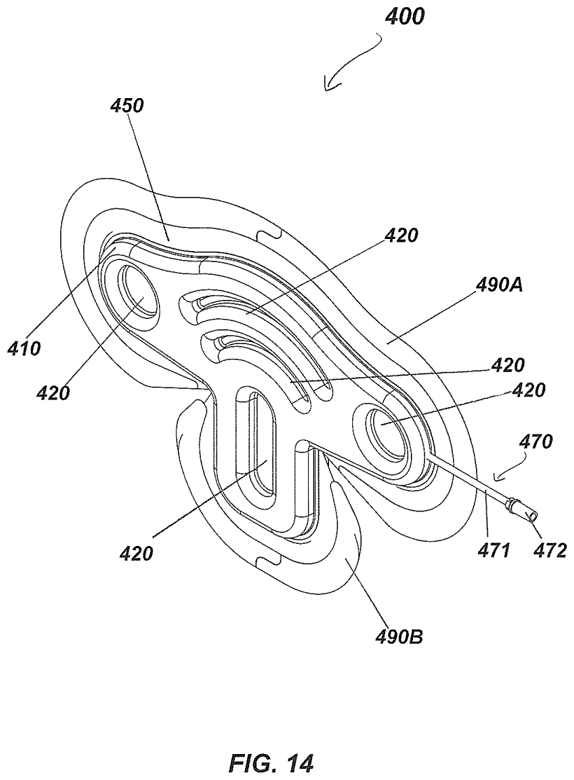

Referring now to FIG. 1, there is illustrated an exemplary embodiment of an apparatus 100 according to the subject invention to elevate a sacrum bone of a user. The apparatus 100 is configured to elevate the sacrum bone when applied to the patient's skin. As shown in FIGS. 1A-1D and 2, this embodiment of the apparatus 100 configured to follow the shape of the bony prominences of a sacrum bone so as to be applied above the buttocks on the back of a user. In this embodiment the shape is similar to a heart with two pre-filled air bladders 110 (110A, 110B) and an opening or aperture 120 configured to insert the bony prominence, in this embodiment the sacrum bone. Other shapes may be employed as well in embodiment not shown such as oval, rectangle, square, circle, crescent, “U” shape or any other known shape in which the sacrum bone can rest while the pre-filled air bladder 110 elevates the bony prominence of the sacrum bone. In another embodiment shown in FIGS. 3A-3D the bladder 100 is not prefilled but instead there is an inflation port 170, for example in this embodiment though not limited to such an inlet valve, in order to inflate the inflatable bladder 110 (110A, 110B, 110C). Also shown in this embodiment is just one inflatable bladder with a connection 110C between the two bladders 110A, 110B. This component can also be employed in the pre-filled air bladders 110A, 110B of FIGS. 1-2, though not shown. The non-limiting exemplary inflation port 170 shown in FIGS. 3A-3D and also in FIG. 14 includes a tube 171 or other connecting means and a valve 172 into which a source of fluid to inflate the inflatable bladder 110 (110A, 110B, 110C).

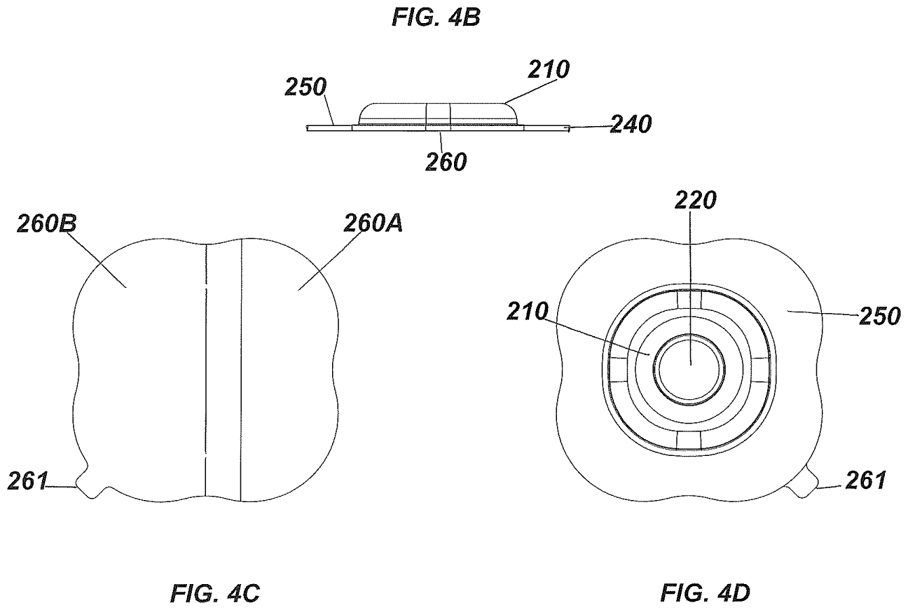

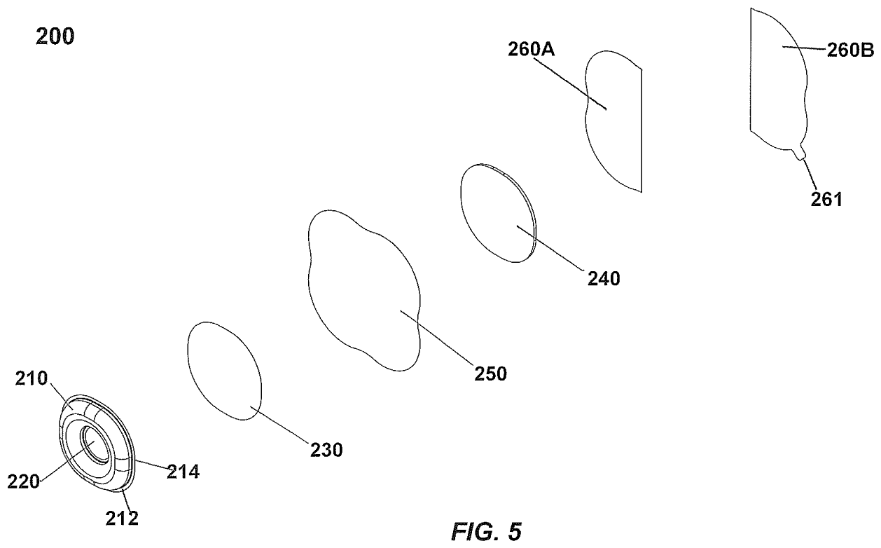

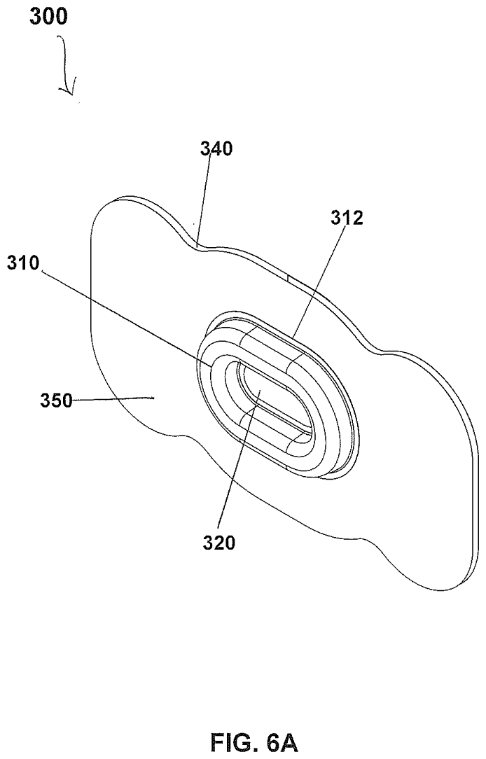

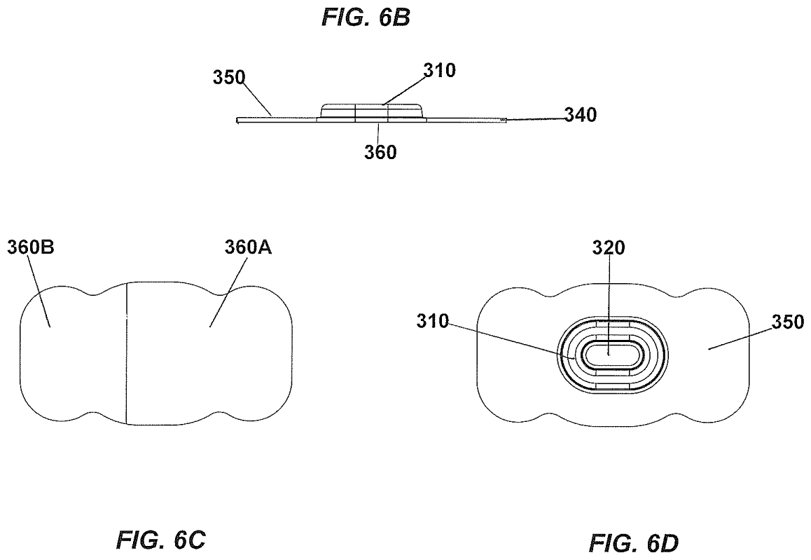

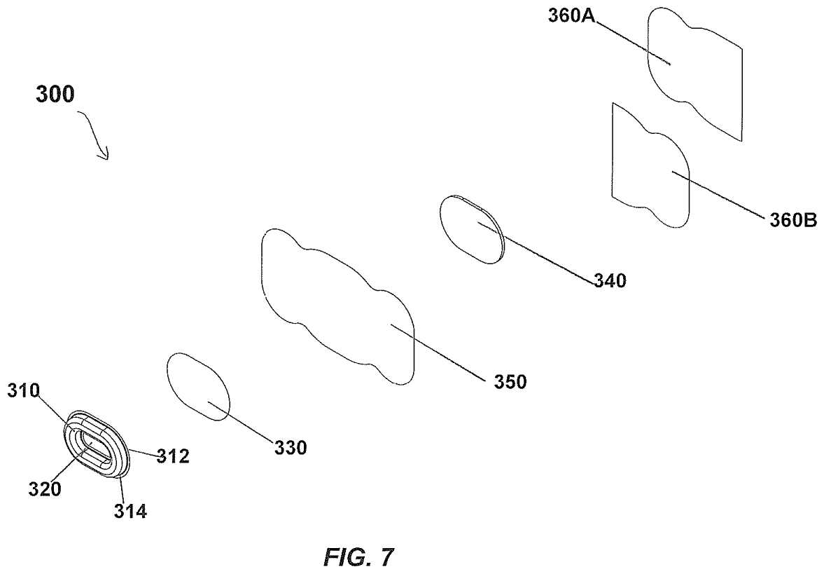

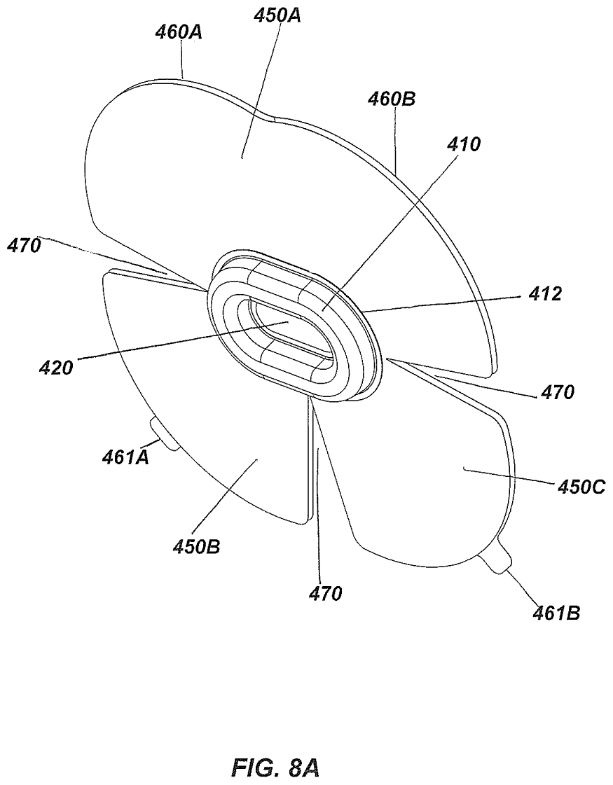

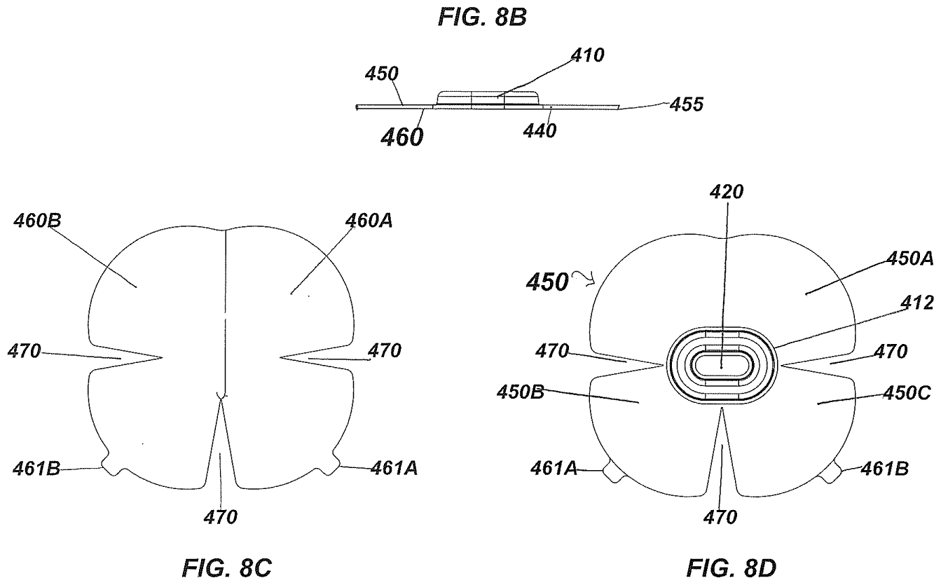

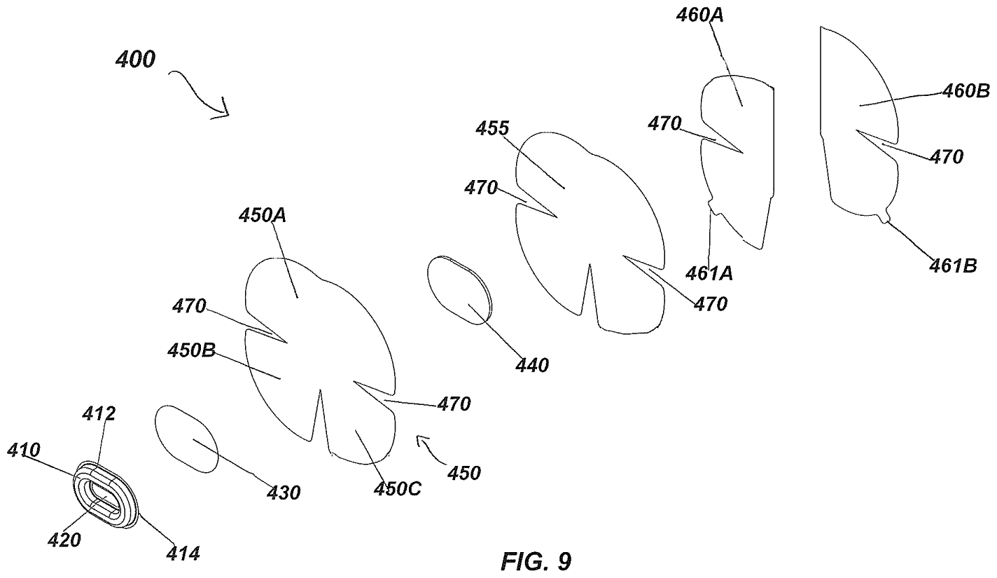

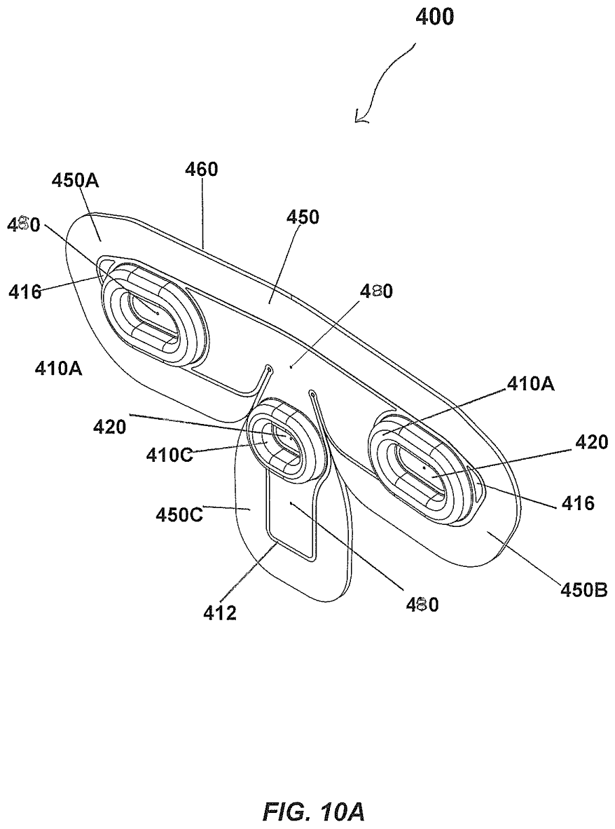

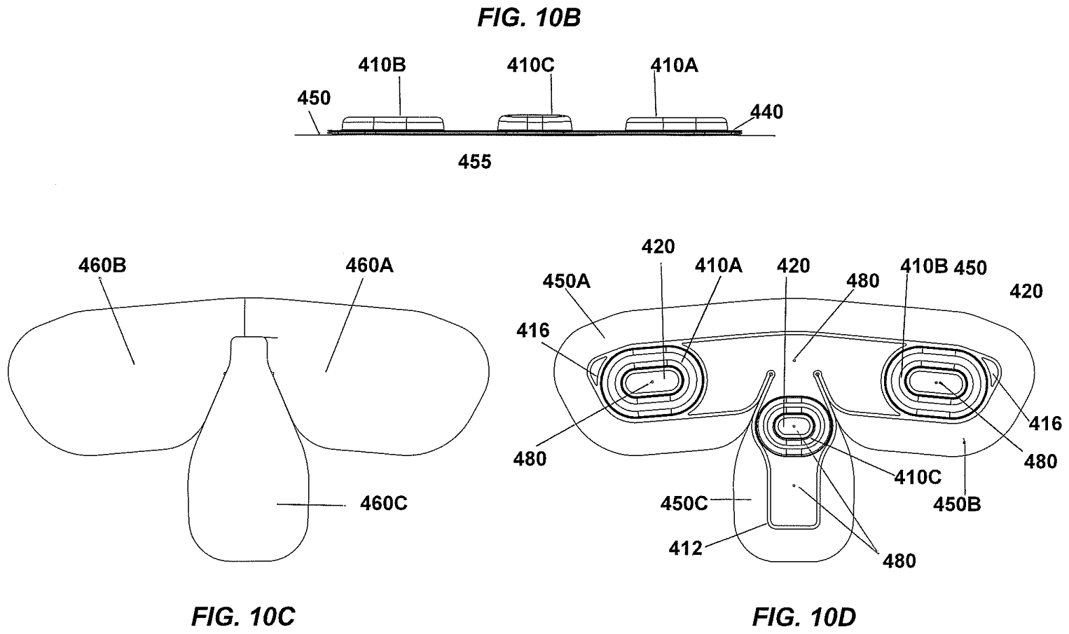

Other bony prominences may be elevated by various embodiments of the current invention such as a hip bone or elbow and one side of an ankle or a heel as shown in FIGS. 4-5, or a metatarsal bone or ball of the foot or other elongated bony prominence as shown in FIGS. 6-7, or the heel as shown in FIGS. 8-9, or a combination of the heel and sides of the ankle as shown in FIGS. 10-14. In some of these embodiments the aperture 120, 220, 320 and 420 are configured for the target bony prominence to rest within the opening or aperture 120, 220, 320, 420 while the bladder 110, 210, 310, 410 elevates the target bony prominence. Various non-limiting exemplary shapes are included such as circles, ovals, rounded rectangles or squares though other shapes may be employed as well in embodiments not shown such as crescent, “U” shape, “V”shape or any other known shape.

In all the embodiments 100, 200, 300, 400 of the present invention which include as shown a pre-filled air bladder 110, 210, 310 410 which is configured to provide elevation to the target bony prominence the pre-filled air bladder 110, 210, 310 410 is formed by joining two sheets, an outer sheet 112, 212, 312, 412 and an inner sheet 114, 214, 314, 414 and in one non-limiting example the air is provided prior to the outer sheet 112, 212, 312, 412 and inner sheet 114, 214, 314, 414 being welded together. In the embodiments including an inflatable bladder 110, 410 the inflation port 170, 470 is connected to the inflatable bladder 110, 410 before or after the outer sheet 112 and inner sheet 114 are joined, such as by welding though other type of inflation ports 170, 470 may be employed. In any of the embodiments the outer sheet 112, 212, 312, 412 and inner sheet 114, 214, 314, 414 may be joined via welding, adhesion, or any other known connecting means. In some embodiments the outer sheet 112 comprises or functions as part of the support for the inventive elevation apparatus as shown in FIGS. 1-14. In the embodiments including an inflatable bladder 110, 410 the inflation port 170, 470 includes a tube 171, 471 and an inlet valve 172, 472 though other types of inflation ports may be employed. The inflation source is not shown though could be a pump such as a hand pump or intermittent pump connected to a valve or hose within the pair bladder. Other inflation means are possible to inflate the inflatable bladder sufficient to elevate the target bony prominence such as a sacrum, hip bone, heel, metatarsal, ankle, shoulder blade or any other body part. A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide inflation within the inflatable bladder so to provide elevation in use by the patient.

In each of the embodiments above the inventive elevation apparatus includes a foam portion 140, 240, 340, 440. The foam portion 140, 240, 340, 440 cushions the body part being elevated by the pre-filled or inflatable air bladder 110, 210, 310, 410. If a pressure ulcer, bed sore or open wound already exists then the foam portion 140, 240, 340, 440 is also capable of absorbing any bleeding or leakage of blood, dye or pus or the like from a pressure ulcer, bed sore or open wound. The foam portion 140, 240, 340, 440 also includes in FIGS. 1-7 and 12-14 an adhesive layer on the skin facing side of the inventive elevation apparatus 100, 200, 300, 400. The adhesive is compatible to the human skin for application of the inventive apparatus 100, 200, 300, 400 to the patient's skin. In other embodiments as shown in FIGS. 8-11, the foam portion 140, 240, 340, 440 is connected to the bladder 110, 210, 310, 410 and optionally the inner sheet 112, 212, 312, 412 or connected to the protective layer 150, 250, 350, 459 via a separate adhesive layer 255, 355. This separate adhesive layer 255, 355 in the embodiments shown in FIGS. 8-11 is the same shape as the hydrophobic layer 150, 250, 350, 450 and in these embodiments they are welded together but any other connecting means may be employed including additional adhesives. The separate adhesive layer 255, 355 may be perforated or not but must be hydrophilic and allow transmission of wound exudate to pass through from the skin and pressure ulcer, bed sore or open wound and into the foam portion 140, 240, 340, 440. By way of example only, the perforations may be holes to enable exudate and fluid to flow through the separate adhesive layer 255, 355. The perforations or holes should be small enough to help prevent tissue ingrowth into the foam portion 140, 240, 340, 440 yet still allow exudate and fluid to flow from the pressure ulcer, bed sore or open wound. The perforations may be slits or holes having a size range of 0,015 mm to 1.5 mm for example. The adhesive on the skin facing portion of the adhesive layer may be any compatible with skin such as by way of example only silicone adhesive with sufficient tack that the inventive elevation apparatus 100, 200, 300, 400 remains on the skin but is removable without trauma to the skin and notably a pressure ulcer or open sore on the skin. Whether the adhesive is on the skin facing side of the foam portion 140, 240, 340, 440 or part of the separate adhesive layer 255, 355, it may be comprised of a pressure sensitive acrylic adhesive or silicone adhesive though could also be comprised of a polymer such as silicone, ethyl vinyl acetate, polyethylene, polypropylene, or polyester, or a combination thereof.

The inventive elevation apparatus 100, 200, 300, 400 may also include a paper, polymer, coating, film or other material which is a protective layer 160, 260, 360, 460 configured to keep the adhesive inactive (whether on the skin side of the foam portion 140, 240, 340, 440 or the additional adhesive layer 255, 355) during shipping and storage but such protective layer 160, 260,360,460 is removed just prior to application onto the skin of the patient. For easier application by the user the film or paper protective layer 160, 260, 360, 460 may be one piece (not shown), two pieces as shown in FIGS. 1-9, three pieces as shown in FIGS. 10-13, or more than four pieces. In some of the embodiments the protective layers 160, 260, 360, 460 have a small tab 161 (161A, 161B), 261, 361, 461 (461A, 461B) which extend beyond the circumference of the inventive elevation apparatus 100, 200, 300, 400 for ease of removing the protective layer 160, 260, 360, 460 by the user.

The hydrophobic layer 150, 250, 350, 450 functions to hold the shape of the elevation apparatus 100, 200, 300, 400 as well as a protection for the user from the exudate within the foam portion 140, 240, 340, 440 transferring to the user's clothing, linens, furniture and the like. The hydrophobic layer 150, 250, 350, 450 may be comprised of film, polyester, or other materials that is liquid-impermeable while still being a breathable moisture-vapor permeable layer so as to allows moisture to evaporate from the inventive elevation apparatus 100, 200, 300, 400. The protective layer 150, 250, 350, 450 shape depends on the target bony prominence being elevated as well as the anatomy of the skin and bones around the bony prominence. For instance, the shape as shown in FIGS. 1-5 is rounded and flared around the respective heart shape 112 for the sacrum bone and circle or rounded square 212 for the hip bone, elbow, should blade, ankle or other bony prominence. In FIGS. 6-7 it is in a rounded and flared rectangle extending beyond the rounded rectangle 312 for the metatarsal, ball of foot or other bony prominence. The heel and ankle shape configurations in FIGS. 8-14 are just three possible variations given the anatomy of the heel and ankle region of the body and other variations may be employed for shapes.

The foam portion 140, 240, 340, 440 may be comprised of any material compatible to the human skin such as by way of example only an open cell polyurethane foam. The foam portion 140, 240, 340, 440 may reduce discomfort to the patient as well as providing absorption of any exudating, bleeding or other oozing liquids on the skin of the patient at the pressure ulcer, bed sore or open wound location. The foam portion 140, 240, 340, 440 may be comprised of a soft, hydrophilic polyurethane foam layer which can function to reduced risk of maceration, exudate any wound liquids which still maintaining comfort to the patient and importantly maintain a moist wound healing environment for the pressure ulcer or skin sore. In some embodiments the foam 140, 240, 340, 440 may be absorbent foam, sponge or dressing (not shown and hereinafter referred to as “the sponge”) which may have the ability to absorb exudate from the wound or ulcer. In addition, the foam or sponge 140, 240, 340, 440 preferably has an open-cell structure which aids in wicking the exudate from the existing pressure ulcer or bed sore. The foam portion 140, 240, 340, 440 may be absorbent, hydrophilic and polymeric and may have a particular range of cell size of 10 microns to 1000 microns. The foam portion 140, 240, 340, 440 is a hydrophilic foam and may be an open celled foam or a mixture of open and closed cells as long as capable of absorbing wound exudate and preferably absorbs the wound exudate rapidly. The faster the foam portion 140, 240, 340, 440 absorbs the exudate then any pooling of blood, pus, and exudate is reduced or even eliminated thereby aiding in healing of the pressure ulcer, bed sore or open wound. The foam portion or sponge 140, 240, 340, 440 may be silicone, hydrophilic, conformable, resilient, and porous allowing exudates to be drawn away from the pressure ulcer, bed sore or open wound and into the inventive elevation apparatus 100, 200, 300, 400. However the material chosen should still maintain a sufficiently moist wound healing environment so as to not dry out the pressure ulcer, bed sore or open wound and retain a balanced moist atmosphere under the inventive elevation apparatus 100, 200, 300, 400. This balance between absorbing the exudate away from the skin surface while maintaining a sufficient moisture level is a component of the material chosen for the foam portion or sponge 140, 240, 340, 440. In some embodiments the foam or sponge 140, 240, 340, 440 may be lightly affixed to the patient's skin by an adhesive film 160, 260, 360, 460 such that the foam or sponge 140, 240, 340, 440 may be peeled off or removed by the user and replaced numerous times if necessary so that it is reusable for a period of time until the foam portion or sponge 140, 240, 340, 440 needs to be replaced because of trapped exudate. Optionally the foam portion or sponge 140, 240, 340, 440 is comprised of an open cell polyurethane foam for balanced absorption and silicone layer for safe application and removal by the user on the skin.

Looking at FIGS. 1-3, the target bony prominence is the sacrum bone. The prefilled air bladder 110 (110A, 110B) is created by connection of the outer sheet 112 and inner sheet 114 as shown in FIGS. 1-2, and in the embodiment where the bladder is inflatable (FIG. 3A-3D) the inflatable bladder 110 (110A, 110B, 110C) is created by connecting of the outer sheet 112 and inner sheet 114 with the optional inflation port 170 also connected during this stage. The formed bladder 110 and outer layer 112 are shaped in this embodiment like a heart and the aperture 120 is configured in the shape of the sacrum bone, though other shapes besides a heart shape may be employed. The shape is also dependent on the size of the sacrum bone for the patient and some ranges of length and width are about 2-8 inches for an adult in width, preferably about 4-7 inches, and about 2-10 inches long, preferably length of about 4-7 inches, with pediatric patients being smaller as needed. The height of the bladder 110 when inflated may be about 0.1-5 inches, preferably about 0.25-2 inches when inflated either pre-filled air bladder or inflatable bladder 110 (110A, 110B, 110C). In one embodiment of formation as shown in FIG. 2, the bladder 110 and outer layer 112 are connected to the hydrophobic layer 150 via an adhesive layer 130. The adhesive layer is not touching the skin so need not be skin compatible. Any known adhesive may be employed and an optional film may be employed to give additional structure to the inventive elevation apparatus 100. In the embodiments shown in FIGS. 1-3, the shape of the hydrophobic layer 150 is rounded and flared and may be dependent on the size of the sacrum bone for the patient and some ranges of length and width are about 3-15 inches for an adult in width, preferably about 5-10 inches, and about 3-15 inches long, preferably length of about 5-10 inches, with pediatric patients being smaller as needed. Where the foam portion or sponge 140 is not the same shape and length and width as the hydrophobic layer 150, the hydrophobic layer also includes a skin compatible adhesive for that portion of the underside which is not covered by the foam portion or sponge 140. The foam portion or sponge 140 is then connected to the underside of the hydrophobic layer 150 via an adhesive, which need not be skin compatible, though the skin facing side of the foam portion or sponge 140 must include a skin compatible adhesive. Lastly, the film or paper protective layer 160 is connected to the under-side adhesive of the foam portion or sponge 140. In use the person removes the film or paper protective layer 160 (160A, 160B) preferably pulling on the tabs 161 (161A, 161B) thereby activating and exposing the adhesive on the foam portion or sponge 140 as well as that portion of the underside of the hydrophobic layer not covered by the foam portion or sponge 140. The inventive elevation apparatus 100 is then placed onto the target bony prominence, in this instance the sacrum bone, with the foam portion or sponge 140 located over any pressure ulcer, bed sore or open wound that exists. The user can then rest in bed with the sacrum bone elevated via the prefilled bladder 110 (110A, 110B) while any exudate from the pressure ulcer, bed sore or open wound is transferred into the foam portion or sponge 140 and the hydrophobic layer 150 protects the user's clothing, linens, furniture and the like from the exudate.

FIGS. 4-5 show an inventive elevation apparatus 200 in the shape of a circle or rounded square and as such the target bony prominence may include a hip bone, elbow, one side of an ankle, should blade or many other bony prominences. In this embodiment the prefilled air bladder 210 is created by connection of the outer sheet 212 and inner sheet 214 as shown in FIG. 5. The formed bladder 210 and outer layer 212 are shaped in this embodiment like a circle or rounded square and is configured to elevate a hip bone, elbow, one side of an ankle, shoulder blade and other bony prominences though other shapes besides a circle or rounded square may be employed. The shape is also dependent on the size of the target bony prominence and some ranges of length and width are about 2-6 inches for an adult in width and length, preferably about 3-5 inches, with pediatric patients being smaller as needed. The height of the prefilled air bladder 210 may be about 0.1-5 inches, preferably about 0.25-2 inches. Not shown is an embodiment where the bladder 210 is inflatable and in such case the height would be similar in range once inflated. In one embodiment of formation as shown in FIG. 5, the bladder 210 and outer layer 212 are connected to the hydrophobic layer 250 via an adhesive layer 230. The adhesive layer is not touching the skin so need not be skin compatible. Any known adhesive may be employed and an optional film may be employed to give additional structure to the inventive elevation apparatus 200. In the embodiments shown in FIGS. 4-5, the shape of the hydrophobic layer 250 is a rounded and flared circle or square and may be dependent on the size of the target bony prominence for the patient and some ranges of length and width are about 3-10 inches for an adult in width and length, preferably about 4-8 inches, with pediatric patients being smaller as needed. Where the foam portion or sponge 240 is not the same shape and length and width as the hydrophobic layer 250, the hydrophobic layer also includes a skin compatible adhesive for that portion of the underside which is not covered by the foam portion or sponge 240. The foam portion or sponge 240 is then connected to the underside of the hydrophobic layer 250 via an adhesive, which need not be skin compatible, though the skin facing side of the foam portion or sponge 240 must include a skin compatible adhesive. Lastly, the film or paper protective layer 260 is connected to the underside adhesive of the foam portion or sponge 240. In use the person removes the film or paper protective layer 260 (260A, 260B) preferably pulling on the tab 261 (not shown may be an embodiment where each of the protective layers 260A, 260B have a tab 261A, 261B) thereby activating and exposing the adhesive on the foam portion or sponge 240 as well as that portion of the underside of the hydrophobic layer not covered by the foam portion or sponge 240. The inventive elevation apparatus 200 is then placed onto the target bony prominence such as the hip bone, elbow, one side of an ankle, shoulder blade and other bony prominences with the foam portion or sponge 240 located over any pressure ulcer, bed sore or open wound that exists. The user can then rest in bed with the hip bone, elbow, one side of an ankle, shoulder blade and other bony prominences elevated via the prefilled bladder 210 while any exudate from the pressure ulcer, bed sore or open wound is transferred into the foam portion or sponge 240 and the hydrophobic layer 250 protects the user's clothing, linens, furniture and the like from the exudate.

FIGS. 6-7 show an inventive elevation apparatus 300 in the shape of a rounded rectangle and as such the target bony prominence may include a ball of a foot, metatarsal or many other bony prominences. In this embodiment the prefilled air bladder 310 is created by connection of the outer sheet 312 and inner sheet 314 as shown in FIG. 7. The formed bladder 310 and outer layer 312 are shaped in this embodiment like a rounded rectangle and is configured to elevate a ball of a foot, metatarsal and other bony prominences though other shapes besides a rounded rectangle may be employed. The shape is also dependent on the size of the target bony prominence and some ranges of length and width are about 0.5-5 inches for an adult in width, preferably about 1-3 inches, and about 0.5-6 inches long, preferably length of about 2-5 inches, with pediatric patients being smaller as needed. The height of the prefilled air bladder 310 may be about 0.1-5 inches, preferably about 0.25-2 inches. Not shown is an embodiment where the bladder 310 is inflatable and in such case the height would be similar in range once inflated. In one embodiment of formation as shown in FIG. 7, the bladder 310 and outer layer 312 are connected to the hydrophobic layer 350 via an adhesive layer 330. The adhesive layer is not touching the skin so need not be skin compatible. Any known adhesive may be employed and an optional film may be employed to give additional structure to the inventive elevation apparatus 300. In the embodiments shown in FIGS. 6-7, the shape of the hydrophobic layer 350 is a rounded and flared rectangle and may be dependent on the size of the target bony prominence for the patient and some ranges of length and width are about 2-10 inches for an adult in width, preferably about 3-6 inches, and about 3-15 inches long, preferably length of about 5-10 inches, with pediatric patients being smaller as needed. Where the foam portion or sponge 340 is not the same shape and length and width as the hydrophobic layer 350, the hydrophobic layer also includes a skin compatible adhesive for that portion of the underside which is not covered by the foam portion or sponge 340. The foam portion or sponge 340 is then connected to the underside of the hydrophobic layer 350 via an adhesive, which need not be skin compatible, though the skin facing side of the foam portion or sponge 340 must include a skin compatible adhesive. Lastly, the film or paper protective layer 360 is connected to the underside adhesive of the foam portion or sponge 340. In use the person removes the film or paper protective layer 360 (360A, 360B; not shown are optional tabs 361 (361A, 361B)) thereby activating and exposing the adhesive on the foam portion or sponge 340 as well as that portion of the underside of the hydrophobic layer not covered by the foam portion or sponge 340. The inventive elevation apparatus 300 is then placed onto the target bony prominence such as the ball of the foot, metatarsal and other bony prominences with the foam portion or sponge 340 located over any pressure ulcer, bed sore or open wound that exists. The user can then rest in bed with the ball of the foot, metatarsal and other bony prominences elevated via the prefilled bladder 310 while any exudate from the pressure ulcer, bed sore or open wound is transferred into the foam portion or sponge 340 and the hydrophobic layer 350 protects the user's clothing, linens, furniture and the like from the exudate.

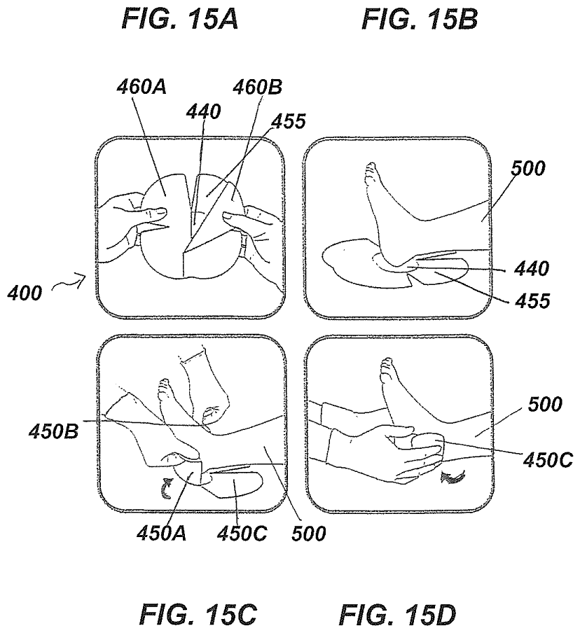

Various embodiments for elevating the heel and/or ankle are shown in FIGS. 8-14. The shape of the hydrophobic layer 450 is different depending on the embodiment chosen, but all elevate the heel and some further elevate the heel and the sides of the ankle. The heel and ankle are some of the most common places for a pressure ulcer, bed sore or open wound and depending on the patient one or more of these exemplary embodiments may be used, as well as other configurations and shapes as long as the heel and/or side of the ankle are elevated to prevent or treat pressure ulcers, bed sores and open wounds on the heel and/or side of the ankle. FIGS. 8-9 and 15 show an inventive elevation apparatus 400 with the prefilled air bladder 410 and outer layer 412 in the shape of a rounded rectangle and the protective layer 450 shaped like a circle or rounded square with certain cut outs or apertures 470 so that the inventive elevation apparatus 400 can adhere to the anatomy of the ankle above the heel. In this embodiment the prefilled air bladder 410 is created by connection of the outer sheet 412 and inner sheet 414 as shown in FIG. 9. The formed bladder 410 and outer layer 412 are shaped in this embodiment like a rounded rectangle and is configured to elevate a heel of a foot though other shapes besides a rounded rectangle may be employed. The shape is also dependent on the size of the target bony prominence and some ranges of length and width are about 0.5-5 inches for an adult in width, preferably about 1-3 inches, and about 0.5-6 inches long, preferably length of about 2-5 inches, with pediatric patients being smaller as needed. The height of the prefilled air bladder 410 may be about 0.1-5 inches, preferably about 0.25-2 inches. Not shown is an embodiment where the bladder 410 is inflatable and in such case the height would be similar in range once inflated. In one embodiment of formation as shown in FIG. 9, the bladder 410 and outer layer 412 are connected to the hydrophobic layer 450 via an adhesive layer 430. The adhesive layer is not touching the skin so need not be skin compatible. Any known adhesive may be employed and an optional film may be employed to give additional structure to the inventive elevation apparatus 400. In the embodiments shown in FIGS. 8-9, the shape of the hydrophobic layer 450 is a rounded and flared circle or square and may be dependent on the size of the target bony prominence for the patient and some ranges of length and width are about 4-15 inches for an adult in width and length, preferably about 6-12 inches, and about 4-15 inches long, preferably length of about 6-12 inches, with pediatric patients being smaller as needed. In this embodiment the foam portion or sponge 440 is set between the hydrophobic layer 450 and a separate adhesive layer 455 and thus the foam portion or sponge need not include a skin compatible adhesive on the underside as the separate adhesive layer 455 has the skin compatible adhesive on its skin facing side. Lastly, the film or paper protective layer 460 (460A, 460B) is connected to the underside adhesive layer 455. As shown in FIG. 15A, in use the person removes the film or paper protective layer 460 (460A, 460B) (not shown in FIG. 15A though shown in FIGS. 8-9 are tabs 461A, 461B) thereby activating and exposing the adhesive on the adhesive layer 455. The user or as shown in FIG. 15B a care giver moves the inventive elevation apparatus 400 under the leg 500 of the patient placing portion 450A under the foot of the user with the foam portion or sponge 440 inventive elevation apparatus 400 placed over the heel with the side portion 450B on the right side of the ankle and portion 450C on the left side of the ankle. As shown in FIG. 15C portion 450A is adhered to the bottom of the foot of the leg 500 with the apertures 470 allowing portions 450B and 450C to remain below the leg 500. Next as shown in FIG. 15D, the care giver, or user, can move up portions 450B and 450C onto the ankle of the user to adhere the elevation apparatus 400 onto the foot of the person. The user can then rest in bed with the heel and other bony prominences elevated via the prefilled bladder 410 while any exudate from the pressure ulcer, bed sore or open wound is transferred into the foam portion or sponge 440 and the hydrophobic layer 450 protects the user's clothing, linens, furniture and the like from the exudate. The angle and length of the apertures or openings or cut out 470 may vary depending on the anatomy of the target location such as the foot, heel and ankle of a leg 500.

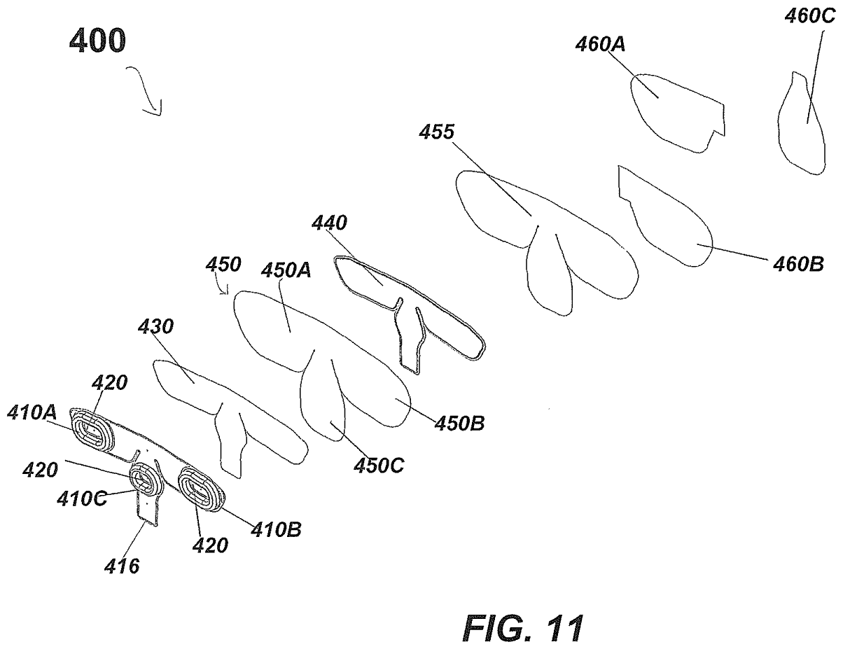

A different shape and configuration is shown in FIGS. 10-11 wherein the target bony prominences are the heel and both sides of the ankle, though the shoulder blades and upper back could also be a target are for this shape configuration, which shape may be a “T” shape”. In this exemplary n inventive elevation apparatus 400 there are three separate prefilled air bladders 410A, 410B, 410C each in the shape of a rounded rectangle and the protective layer 450 is shaped like a circle rectangle with another rounded rectangle in the middle at a right angle, so that the inventive elevation apparatus 400 can adhere to the anatomy of the ankle above the heel while elevating the heel and both sides of the ankle. In this embodiment the three prefilled air bladders 410A, 410B, 410C are created by connection of the outer sheet 412 and inner sheet 414 as shown in FIG. 11 with the entire form noted and include certain through holes 480, functioning both as air venting and for alignment in manufacture, and also include corner weld lines 416 for better support. The formed bladders 410A, 410B, 410C are shaped in this embodiment like a rounded rectangle and is configured to elevate a heel of a foot and each side of the ankle, though other shapes besides a rounded rectangle may be employed. The shape is also dependent on the size of the target bony prominence and some ranges of length and width are about 0.5-5 inches for an adult in width, preferably about 1-3 inches, and about 0.5-6 inches long, preferably length of about 2-5 inches, with pediatric patients being smaller as needed. The height of the prefilled air bladders 410A, 410B, 410C may be about 0.1-5 inches, preferably about 0.25-2 inches. Not shown is an embodiment where the bladders 410 are inflatable via an inflation port and possibly connected to one another or maybe only two connected to another bladder 410 and in such case the height would be similar in range once inflated. In one embodiment of formation as shown in FIG. 11, the prefilled air bladders 410A, 410B, 410C and outer layer 412 and formation 416 are connected to the hydrophobic layer 450 via an adhesive layer 430. The adhesive layer is not touching the skin so need not be skin compatible. Any known adhesive may be employed and an optional film may be employed to give additional structure to the inventive elevation apparatus 400. In the embodiments shown in FIGS. 10-11, the shape of the hydrophobic layer 450 is a rounded rectangle with another rounded rectangle located in the middle at a right ankle so there are three portions referenced as 450A, 450B, 450C each having one prefilled air bladder 410 thereon (410A, 410B, 410C). The size ranges for this embodiment may vary dependent on the size of the target bony prominence for the patient and some ranges of length and width are about 5-15 inches for an adult in width, preferably about 6-12 inches wide, and about 8-25 inches long, preferably length of about 10-20 inches long, with pediatric patients being smaller as needed. In this embodiment the foam portion or sponge 440 is in the shape or a sounded rectangle between two prefilled air bladders 410A, 410B and then another rounded rectangle at a right angle in the center of the long portion including the third prefilled air bladder 410C. The foam portion or sponge itself has a range of shape of about 3-12 inches for an adult in width, preferably about 4-10 inches wide, and about 6-20 inches long, preferably length of about 8-18 inches long, with pediatric patients being smaller as needed. The foam portion or sponge 440 is set between the hydrophobic layer 450 and a separate adhesive layer 455 and thus the foam portion or sponge 440 need not include a skin compatible adhesive on the underside as the separate adhesive layer 455 has the skin compatible adhesive on its skin facing side. Lastly, the film or paper protective layer 460 (460A, 460B, 460C) is connected to the underside of the adhesive layer 455. In use the person removes the film or paper protective layer 460 (460A, 460B, 460C) (thereby activating and exposing the adhesive on the adhesive layer 455. The user moves the inventive elevation apparatus 400 under the leg 500 of the patient placing portion 450C under the foot of the user with prefilled air bladder 410C over the heel and the places portion 450A on the left side of the ankle with prefilled air bladder 410A over the bony protruding part of the ankle and finally places portion 450B on the right side of the ankle with the prefilled air bladder 410B placed over the bony protruding part of the right ankle. The foam portion or sponge 440 inventive elevation apparatus 400 placed over the heel with the side portion 450B on the right side of the ankle and portion 450C on the left side of the ankle. As shown in FIG. 15C portion 450A is adhered to the bottom of the foot of the leg the user can then rest in bed with the heel and other bony prominences elevated via the three prefilled air bladders 410A, 410B, 410C while any exudate from the pressure ulcer, bed sore or open wound is transferred into the foam portion or sponge 440 and the hydrophobic layer 450 protects the user's clothing, linens, furniture and the like from the exudate.

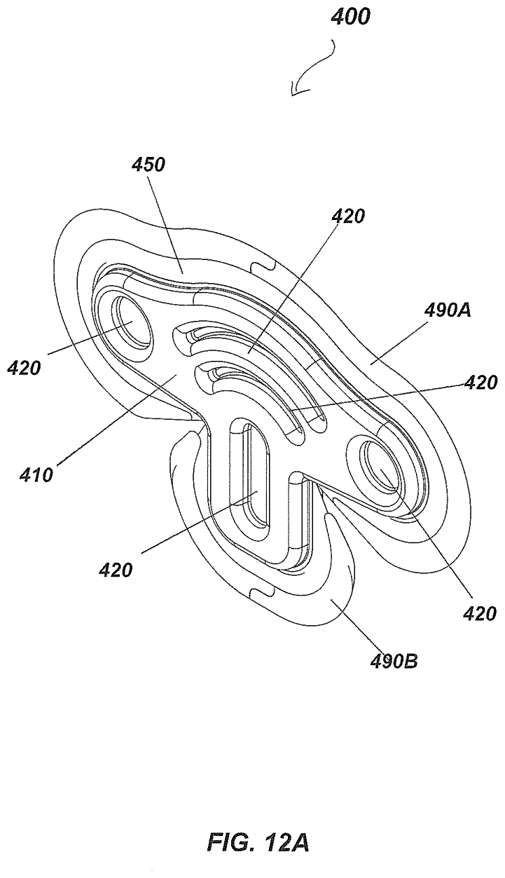

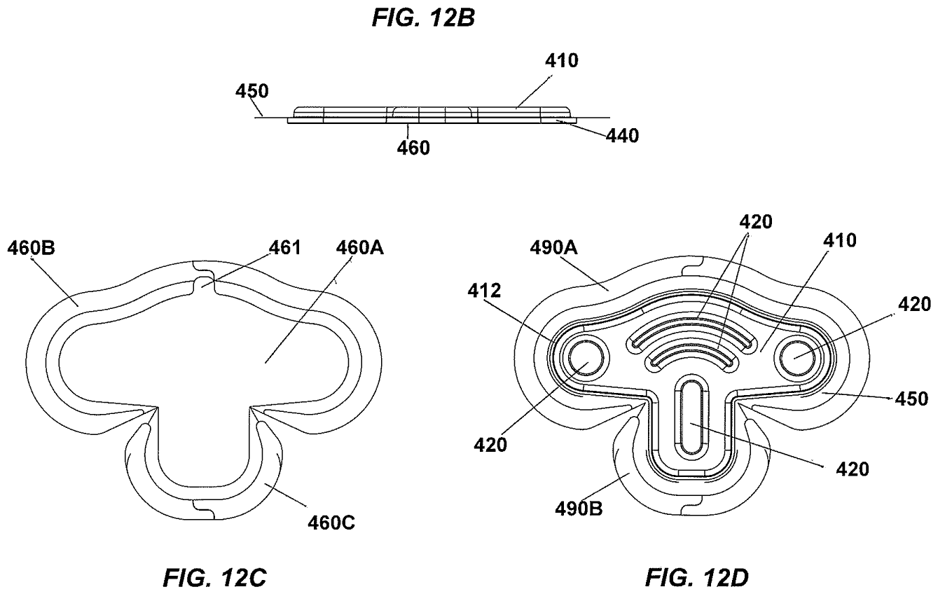

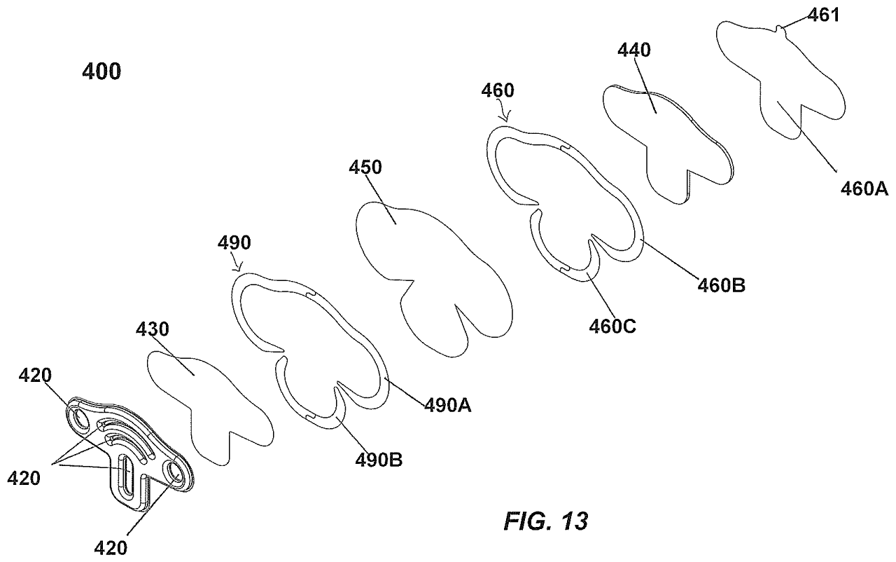

Yet another embodiment is shown in FIGS. 12-14 to elevate the bony prominences of the heel and both sides of the ankle. The prefilled air bladder 410 is created by connection of the outer sheet 412 and inner sheet 414 as shown in FIG. 13, and in the embodiment where the bladder is inflatable (FIG. 14) the inflatable bladder 410 is created by connecting of the outer sheet 412 and inner sheet 414 with the optional inflation port 470 also connected during this stage. In this embodiment there is only one bladder 410 with multiple apertures of openings 420 for both bony prominences to rest as well as provide support and movement of the bladder 410 when adhered to the foot of the user. The formed bladder 410 and outer layer 412 are shaped in this embodiment with a rounded rectangle and a second rounded rectangle at the center of the first at a right angle, though other shapes besides a “T” shape may be employed. The shape is also dependent on the size of the foot and lower leg for the patient and some ranges of length and width are about 2-20 inches for an adult in width, preferably about 4-15 inches wide, and about 2-25 inches long, preferably length of about 4-15 inches, with pediatric patients being smaller as needed. The height of the bladder 410 when inflated may be about 0.1-5 inches, preferably about 0.25-2 inches when inflated either pre-filled air bladder or inflatable bladder 410. In one embodiment of formation as shown in FIG. 13, the bladder 410 and outer layer 412 are connected to a stiffener or support layer 490 (490A, 490B) which functions to support the elevation apparatus 400 as the bladder 410 is the entire width and length of the inventive elevation apparatus 400. The stiffener or support layer 490 may be in one piece or as shown in this embodiment two pieces with a small gap between the horizontal portion of the blabber 410 (portion 490A) and vertical portion of the bladder (portion 490B). The stiffener or support layer is in the shape of the outer circumference of the bladder 410 and will have contact with the skin of the user. The hydrophobic layer 450 is then connected to the stiffener or support layer 490 via an adhesive layer 430. The adhesive layer 430 is not touching the skin so need not be skin compatible. Any known adhesive may be employed and an optional film may be employed to give additional structure to the inventive elevation apparatus 400. Where the foam portion or sponge 440 is not the same shape and length and width as the hydrophobic layer 450, the hydrophobic layer also includes a skin compatible adhesive for that portion of the underside which is not covered by the foam portion or sponge 440. The foam portion or sponge 440 is then connected to the underside of the hydrophobic layer 450 via an adhesive, which need not be skin compatible, though the skin facing side of the foam portion or sponge 440 must include a skin compatible adhesive. Lastly, the film or paper protective layer 460 is connected to the underside adhesive of the foam portion or sponge 440. In use the person removes the film or paper protective layer 460 (460A, 460B) preferably pulling on the tab 461 and then removing portions 460B and 460, thereby activating and exposing the adhesive on the foam portion or sponge 440 as well as that portion of the underside of the hydrophobic layer not covered by the foam portion or sponge 440 and any exposed portion of the stiffener or support layer 490 (490A, 490B). The inventive elevation apparatus 400 is then placed onto the target bony prominence, in this instance the heel and ankle, with the vertical portion (closer 490B with the aperture 420 in the shape of a rounded rectangle) placed under the foot and heel of the user, and then the two apertures 420 in the shape of circles placed over the bony protruding part of each side of the ankle. The inner aperture 420 function for movement of the ankle when the inventive elevation apparatus 400 is adhered to the skin of the user. The user can then rest in bed with the foot, heel and both side of the ankle sacrum bone elevated via the prefilled or inflated bladder 410 while any exudate from the pressure ulcer, bed sore or open wound is transferred into the foam portion or sponge 440 and the hydrophobic layer 450 protects the user's clothing, linens, furniture and the like from the exudate.

The inventive apparatus 100, 200, 300, 400 is configured to apply localized elevation and reduce pressure, friction and shear and therefore treat, reduce or even to prevent pressure ulcers, bed sores and open wound to the user when immobile or while moving and rubbing against clothing, linens, furniture and the like. The pre-filled air bladder 110, 210, 310, 410 is a set height over the target bony prominence such as the metatarsal, foot bones, ankle, heel, hip bone, elbow, shoulder blades or tailbone/sacrum so as to elevate the bony prominence when the user is resting that body part on a chair or bed or another surface. The air bladder may also be provided in an un-inflated state and then inflated via an inflation means after the inventive elevation apparatus is applied to the skin of the user.

Other embodiments now shown may include a suction conduit for example provided in between the foam 140, 240, 340, 440 and the skin with the suction conduit may be oriented downwardly towards the sponge so that when a source of negative pressure is applied to the suction conduit, that negative pressure is applied to pressure ulcer via the sponge. As a result, exudate can be wicked and suctioned away from the pressure ulcer or bed sore via the sponge and into the conduit (such as a tube and other devices). In one embodiment, the conduit may be easily detached from the inventive apparatus 100, 200, 300, 400 so that the patient can walk or be transported away from the source of suction while pressure is still being applied to the pressure ulcer or bed sore.

Certain target bony prominences include elevating and exudating the heel and/or ankle while the user is in bed and resting on either side of the ankle and alleviating stress on the Achilles tendon. This portion of the ankle and lower leg is prone to pressure ulcers and bed sores and needs elevation to treat, heal and prevent injury. In use the pre-filled air bladder 410 (410A, 410B, 410C) elevates the bony prominences of the ankle and heel above the bed sheet or chair or other place where the ankle and heel would lie and reduces pressure on such skin location, thereby treating, reducing and even preventing pressure ulcers and bed sores. Other target bony prominences include the sacrum bone and hip bone which if elevated with the inventive elevation apparatus 100, 200 may reduce, treat and even prevent pressure ulcers and bed sores at the hip bone and sacral location. Indeed, the level of pressure felt by the patient at the pressure ulcer, bed sore or open wound site is reduced. Not shown but contemplated are optional medications or treatments included in the skin-contact adhesive layer such as silver, zinc oxide, antibiotics, pain killers and the like. Also not included but contemplated are sensors that alert the user to when the foam portion or sponge 140, 240, 340, 440 are filled with exudate and the elevation apparatus 100, 200, 300, 400 needs to be changed.

The present invention of the elevation apparatus 1 00, 200, 300, 400 solve many problems associated with pressure ulcers, bed sores and open wounds. To prevent pressure ulcers and bed sores from happening, the present invention includes a foam application which assists to control bleeding and other bodily fluids from leaking and the improvement is to elevate bony prominences from the bed, chair, couch or other place where the individual is resting. Adding air bladders, pre-filled or ability to be filled, thus elevates the bony prominences from the bed, chair, couch or other location where the body part is resting. This can be used to reduce pain and suffering but also economic strain on nursing homes, assisted living facilities, hospitals and clinics after surgery or other reasons while a user may be bed ridden for a period of time. Again, the elevation around bony prominences reduces ulcers, bed sores and open wounds at the skin on the bony prominences which has reduced probability of forming an ulcer thereon.

The present invention has been illustrated and described with respect to specific embodiments thereof, which embodiments are exemplary and illustrative of the principles of the invention and are not intended to be exclusive or otherwise limiting embodiments. The system or kit of the invention can include a compression wrap such as that co-owned by Applicant known as the Aero-Wrap, Application Serial No. 16/328718, and older compression wraps as those disclosed in co-owned (and incorporated herein as reference) in U.S. Pat. Nos. 9,033,906 and 7,967,766 and 7,559,908 and U.S. Ser. No. 13/444,600. All of these compression wraps may be placed over the inventive elevation apparatus and further treat the pressure ulcers, bed sores and open wounds.

Further, while in the foregoing embodiments additional components may be implemented using any of a variety of preformed and/or prefilled cushioning materials such as foam cushions and/or air, gel, or other fluid filled non-inflatable cushions. Further, while particular shapes, sizes, and materials have been described for purposes of illustration, it will be recognized that any of a variety of shape or size can be used, and the materials described are not exclusive but merely illustrative. Also, as noted above, while the bladder shown is inflated with air, it will be appreciated that any other fluid or medium such as liquid or gel can be used. Moreover, as also noted, it will be understood that bladders may be configured to have multiple pneumatically independent and/or pneumatically coupled bladder sections, and may also be configured to have various contours or lobulations. Also, the foam and adhesive layer can include medications to treat the current pressure ulcers and sores such as anti-inflammatory, medication to reduce or treat infections, medications to reduce or treat pain at the location, and any other medication or nutrients to be applied at the pressure ulcer and bed sore location injury. Other additions are possible and encompassed in this invention.

While the subject invention of the present disclosure has been described with respect to preferred and exemplary embodiments, those skilled in the art will readily appreciate that various changes and/or modifications can be made to the invention without departing from the spirit or scope of the invention as described herein. There have been described and illustrated herein several embodiments of an intermittent pressure apparatus and a method of installing and operating same. While particular embodiments of the invention have been described, it is not intended that the invention be limited thereto, as it is intended that the invention be as broad in scope as the art will allow and that the specification be read likewise. Thus, while particular shapes and sizes of inflatable bladders and straps have been disclosed, it will be appreciated that other shapes, sizes, and attachment means may be used as well. It will also be understood that while adhesive means have been disclosed for helping to secure the skin, other types of attachments such as wraps may be used. It will therefore be appreciated by those skilled in the art that yet other modifications could be made to the provided invention without deviating from its spirit and scope as claimed.

I claim:

1. An elevation apparatus to elevate a bony prominence on a user, comprising:
a permeable skin-compatible adhesive layer;
an absorbent layer for absorbing wound exudate;
a hydrophobic layer above the absorbent layer; and
at least one prefilled air bladder connected to the hydrophobic layer including at least one aperture configured for placement over the bony prominence being elevated.

2. The elevation apparatus of claim 1, wherein the absorbent layer is a foam or sponge.

3. The elevation apparatus of claim 2, wherein the absorbent layer is selected from an open cell foam, closed cell foam, partial open and closed cell foam, silicone foam, and combinations thereof.

4. The elevation apparatus of claim 1, wherein the absorbent layer either has the permeable skin-compatible adhesive layer therein is a foam or is set between the permeable skin-compatible adhesive layer and the hydrophobic layer.

5. The elevation apparatus of claim 1, wherein the bony prominence is selected from the group consisting of a heel, metatarsal, ankle, elbow, hip, should blade or sacrum.

6. The elevation apparatus of claim 1, wherein the at least one prefilled air bladder includes at least one aperture and the prefilled air bladder and aperture are configured to be placed over a heel of a user.

7. The elevation apparatus of claim 1, wherein the at least one prefilled air bladder includes at least one aperture and the prefilled air bladder and aperture are configured to be placed over a hip bone or elbow of a user.

8. The elevation apparatus of claim 1, wherein the at least one prefilled air bladder includes at least one aperture and the prefilled air bladder and aperture are configured to be placed over a metatarsal bone of a user.

9. The elevation apparatus of claim 1, wherein the at least one prefilled air bladder includes at least three apertures and the prefilled air bladder and apertures are configured to be placed over a heel and both sides of an ankle of a user.

10. The elevation apparatus of claim 1, further comprising a second and a third prefilled air bladders, each of the three prefilled air bladders including one aperture and the three prefilled air bladders and apertures are configured to be placed over a heel and both sides of an ankle of a user.

11. The elevation apparatus of claim 1, further comprising a second prefilled air bladder both creating one aperture and the two prefilled air bladders and one aperture are configured to be placed over a sacrum bone of a user.

12. The elevation apparatus of claim 1, wherein the shape of the elevation apparatus is selected from the group consisting of circle, square, rectangle, heart, rounded square, rounded rectangle, “T” shape, “U” shape, “V” shape and combinations thereof.

13. The elevation apparatus of claim 1, further comprising medication, silver, zinc oxide, antibiotics, pain killers and the like impregnated into the permeable skin-compatible adhesive layer.

14. The elevation apparatus of claim 1 wherein the bony prominence is a hip bone or elbow of a user, the at least one prefilled air bladder is in the shape of a circle or rounded square having a length and width of about 2 inches to about 6 inches and the hydrophobic layer is in the shape of a circle or rounded square having a length and width of about 3 inches to about 15 inches.

15. The elevation apparatus of claim 1 wherein the bony prominence is a metatarsal bone or ball of a foot of a user, the at least one prefilled air bladder is in the shape of a rounded rectangle having a length of about 0.5 inches to about 5 inches and a width of about 0.5 inches to about 6 inches and the hydrophobic layer is in the shape of a rounded rectangle having a length of about 1 inches to about 10 inches and a width of about 1 inch to about 10 inches.

16. The elevation apparatus of claim 1 wherein the bony prominence is a combination of a heel and the at least one air bladder is in the shape of a rounded rectangle having a length of about 0.5 inches to about 6 inches and a width of about 0.5 inches to about 5 inches and the hydrophobic layer is in the shape of a circle or rounded square having a length and width of about 4 inches to about 15 inches, further comprising at least three slits in the hydrophobic layer configured for adherence to the side of the ankle of a user.

17. The elevation apparatus of claim 1 wherein the bony prominence is a combination of a heel and both sides of an ankle, further comprising a second and a third prefilled air bladder each having a length of about 0.5 inches to about 6 inches and a width of about 0.5 inches to about 5 inches, and the absorbent layer is in the shape of a “T” having a length of about 6 inches to about 20 inches and a width of about 3 inches to about 12 inches, and the hydrophobic layer is in the shape of a “T” having a width of about 6 inches to about 12 inches and a length of about 8 inches to about 25 inches.

18. The elevation apparatus of claim 1 wherein the bony prominence is the sacrum bone, further comprising a second air bladder and the hydrophobic layer is in the shape of a heart or rounded heart, square or circle having a length and width of about 2 inches to about 10 inches.

19. The elevation apparatus of claim 1 wherein the bony prominence is a hip bone or elbow of a user, the at least one prefilled air bladder is in the shape of a circle or rounded square having the length and width of about 2 inches to about 6 inches and the hydrophobic layer is in the shape of a circle or rounded square having the length and width of about 2 inches to about 10 inches.

20. The elevation apparatus of claim 1 further comprising a second prefilled air bladder in connection with the first prefilled air bladder.

21. The elevation apparatus of claim 1 further comprising a protective layer over the permeable skin-compatible adhesive layer, wherein the protective layer is comprised of a film, polymer, paper or combinations thereof.

22. An elevation apparatus to elevate a bony prominence on a user, comprising:
a permeable skin-compatible adhesive layer;
an absorbent layer for absorbing wound exudate;
a hydrophobic layer above the absorbent layer; and
at least one inflatable air bladder capable of inflation via an inflation port and a inflation means, the at least one inflatable air bladder connected to the hydrophobic layer including at least one aperture configured for placement over the bony prominence being elevated.

23. The elevation apparatus of claim 22, wherein the absorbent layer is selected from the group consisting of a foam, sponge, open cell foam, closed cell foam, partial open and closed cell foam, silicone foam, and combinations thereof.

24. The elevation apparatus of claim 22, wherein the absorbent layer either has the permeable skin-compatible adhesive layer therein is a foam or is set between the permeable skin-compatible adhesive layer and the hydrophobic layer.

25. The elevation apparatus of claim 22, wherein the bony prominence is selected from the group consisting of a heel, metatarsal, ankle, elbow, hip, should blade or sacrum.

26. A method of elevating a bony prominence of a user, comprising:
taking an elevation apparatus from its packing, the elevation apparatus including at least one prefilled air bladder, a hydrophobic layer, an absorbent layer and a permeable skin-compatible adhesive layer;
removing the protective layer comprised of film, polymer, paper or combination thereof thereby activating the permeable skin-compatible adhesive layer below the protective layer on the elevation apparatus; and
placing the at least one prefilled air bladder and absorbent layer for absorbing wound exudate over the bony prominence and pressing on the elevation apparatus so that the bony prominence is in contact with the permeable skin-compatible adhesive layer.
